(19) United States
(12) Patent Application Publication
(10) Pub. No.: US 2005/0107753 A1
(43) Pub. Date: May 19, 2005

(54) Microinfusion device

(76) Inventors: Ali Rezai, Bratenhal, OH (US); Ashwini Sharan, Mt Laurel, NJ (US)

Correspondence Address: KENYON & KENYON, ONE BROADWAY, NEW YORK, NY, 10004, US

(21) Appl. No.: 10/502,661

(22) PCT Filed: Jan. 31, 2003
(86) PCT No.: PCT/US03/02843

Related U.S. Application Data
(60) Provisional application No. 60/353,706, filed on Feb. 1, 2002.
(60) Provisional application No. 60/358,176, filed on Feb. 20, 2002.

(51) Int. Cl. A61M 31/00

(52) U.S. Cl. 604/288.04; 604/891.1

(57) ABSTRACT

A microinfusion device, comprising: (a) a subcutaneously implantable reservoir configured to contain a drug, the reservoir being mountable within a burr hole of a skull of a subject; (b) a dose control system configured to control flow of the drug; and (c) a microcatheter to deliver the drug from the reservoir to a target location. Another embodiment provides a microinfusion device, comprising: a subcutaneously implantable reservoir configured to contain a drug, the implantable reservoir having at least two outlets. A further embodiment provides a microinfusion device, comprising: (a) a subcutaneously implantable reservoir configured to contain a drug, the reservoir being mountable within a burr hole in a skull of a patient, the reservoir being ring-shaped; and (b) a microcatheter configured to deliver the drug from the reservoir to a target location. Yet another embodiment provides a microinfusion device, comprising: (a) a subcutaneously implantable reservoir having septations configured to separate different drugs within the reservoir; and (b) at least one microcatheter configured to deliver the different drugs from the reservoir to at least one target location.

### CROSS-REFERENCE TO RELATED APPLICATIONS

This application claims the benefit under 35 U.S.C. § 119(e) of U.S. provisional application Ser. No. 60/353,706 filed Feb. 1, 2002, and U.S. provisional application Ser. No. 60/358,176, filed Feb. 20, 2002, the entire contents of which are hereby incorporated herein by reference in their entirety.

### BACKGROUND

The present invention relates generally to controlled application of medication, electrical stimulation, or both in a desired area. It finds particular application in conjunction with both microinfusion and neurostimulator systems and will be described with particular reference thereto.

Presently, infusion systems include a fully implantable pump which is capable of delivering drugs into the intrathecal space. The most common application for this device is for the delivery of intrathecal baclofen, or a variety of other intrathecal pain medications. Recently, a pump has been developed for delivery of insulin although this type of pump has not been used to deliver drugs to the central nervous system. This existing technology involves the invasive implantation of an expensive and bulky pump system.

Currently commercially available percutaneous testing electrical stimulation devices extend out of the skin and, thus, can only be used for a short duration, typically less than two weeks and most commonly about one week. A major reason for the limited duration is the increased risk of infection. Once the trial period is over, the extension through the skin is cut or otherwise removed. Nevertheless, presently used electrical stimulation devices are suspected to increase the infectious risk for the permanent implant.

Existing electrical stimulation technology generally requires an implanted pulse generator or IPG. Such devices are bulky, expensive and in many cases, nonrechargeable.

### SUMMARY

In one example embodiment of the present invention, an infusion device, e.g., a microinfusion device, is provided which includes a chamber for allowing the introduction of at least one medication into the central or peripheral nervous system (e.g., the brain) with a microcatheter.

In one example embodiment of the present invention, a microinfusion device is provided, comprising: (a) a subcutaneously implantable reservoir configured to contain a drug, the reservoir being mountable within a burr hole of a skull of a subject; (b) a dose control system configured to control flow of the drug; and (c) a microcatheter configured to deliver the drug from the reservoir to a target location.

In another example embodiment, a microinfusion device is provided, comprising: a subcutaneously implantable reservoir configured to contain a drug, the implantable reservoir having at least two outlets. The device may further include a dose control system, as described below. The device may further include respective microcatheters connected to each of the at least two outlets, wherein at least one of the respective microcatheters is connected to a reservoir infusion system, said reservoir infusion system being implantable within the body of a subject to provide a source of the drug, and the at least another of the respective microcatheters is configured to deliver the drug to a target location. In one example embodiment, the device may further include a sensor at the distal end of the at least second microcatheter to provide feedback to the dose control system. Sensors which may be used in any of the devices provided herein are described below.

In a further example embodiment of the present invention, a microinfusion device is provided, comprising: (a) a subcutaneously implantable reservoir configured to contain a drug, the reservoir being mountable within a burr hole in a skull of a patient, the reservoir being ring-shaped; and (b) a microcatheter configured to deliver the drug from the reservoir to a target location. In one preferred embodiment, the device is a burr hole ring which does not require attachment to a burr hole device, but may be directly mounted within a burr hole within a skull of a subject. The device may be configured to engage or be mounted within a burr hole ring or device, as described below. The device may further include any of a dose control system configured to control flow of the drug and a sensor, as described below. In another embodiment, the reservoir may have at least one notch for insertion of a deep brain stimulation (DBS) electrode through the at least one microcatheter.

In yet another example embodiment of the present invention, a microinfusion device is provided, comprising: (a) a subcutaneously implantable reservoir having septations configured to separate different drugs within the reservoir; and (b) at least one microcatheter configured to deliver the different drugs from the reservoir to at least one target location. The device may be directly mounted within a burr hole within a skull of a subject, i.e., it is a burr hole device/ring. The device may also be configured to engage or be mounted within a burr hole ring/device, and may further include a dose control system configured to control flow of the drug and a sensor. In another aspect, the reservoir may also have at least one notch for insertion of a deep brain stimulation electrode through the microcatheter.

In one example embodiment of the present invention, a drug or medication delivery device is provided, comprising: a reservoir configured to contain a drug, and a receiver configured to wirelessly receive signals and to control a dosing of the drug in accordance therewith. The signals may include, for example, radio frequency (RF) signals. Both the reservoir and the receiver may be subcutaneously implantable. The delivery device may include a microcatheter configured to deliver the drug to a target location, and a valve system coupled to the microcatheter and configured to control the dosing of the drug as a function of the signals. The receiver may include coils and/or antennas. The receiver may be an active or a passive device.

The example infusion devices may include a DBS electrode to provide neurostimulation, in addition to microinfusion of drug(s) to target locations in the nervous system. Devices including such electrodes may be controlled by radio frequence (RF). In an example embodiment, as described below in greater detail, radio frequency coils or antennas configured to receive signals from an external controller, the dose control system controlling the flow of the drug in accordance with the received signals. Such devices may thus obviate the current use of implantable pulse generators (IPGs).

In accordance with another aspect of the present invention, the example microinfusion devices may include radio frequency (RF) coils or antennas disposed proximate to or externally on the reservoir, wherein the radio frequency coils or antennas are configured to receive signals from an external controller, the dose control system controlling the flow of the drug in accordance with the received signals. Microinfusion of the drug(s) may thus be controlled by radio frequency. The radio frequency coils or antennas may receive signals from the external controller to adjust and/or control dosing.

In accordance with another aspect of the present invention, the microinfusion device is attached to or is an integral part of a deep brain burr device. In example embodiments of the above-described devices, the reservoir may be mounted within a burr hole ring, which is alternatively called a burr hole device herein.

In further example embodiments of the present invention, each microinfusion device may include a dose control system. The dose control system may include a valve system between the reservoir and the microcatheter for flowing of the drug. In accordance with another aspect of the present invention, the device may include a valve system permitting predetermined dosing of the medication. The valve system may either be fixed for delivery of the drug or electronically adjustable. The drug to be delivered may a predetermined dose. An electronically adjustable valve in this system may permit control of delivery of the drug, whose dose need not be predetermined prior to being contained in the reservoir. In one embodiment, the valve system may be controlled via RF signals.

In another example of the present invention, each device may include a dose control system, wherein the dose control system includes a ball-bearing system. The ball-bearing system may be magnetically adjustable for delivery of the drug. Alternatively, the ball-bearing system may be electronically adjustable to control delivery of the drug. The drug contained in the reservoir may be in a predetermined or non-predetermined dose.

Alternate embodiments of the devices described herein may include a propulsion system in the reservoir, which system utilizes osmosis or gravity to flow the drug from the reservoir.

In another embodiment of the present invention, a subcutaneously implantable neurostimulator is provided. In one embodiment, antennas or coils are mounted subcutaneously in a burr hole ring. The coils or antennas are coupled to a neurostimulation or a brain stimulation electrode (e.g., a DBS electrode).

In another example embodiment, a drug delivery device is provided, which includes a reservoir configured to contain a drug; and a receiver configured to wirelessly receive signals and to control a dosing of the drug in accordance therewith. The signals may be radio frequency signals. The reservoir may be subcutaneously implantable. In one embodiment, the device includes a dose control system configured to control the dosing of the drug as a function of the signals. The dose control system may include, for example, a valve system. In another embodiment, the device may include a microcatheter coupled to the reservoir and configured to deliver the drug to the target location.

Further aspects and advantages of the present invention will become apparent to those of ordinary skill in the art upon reading and understanding the following Detailed Description.

### BRIEF DESCRIPTION OF THE DRAWINGS

The present invention may take form in various components and arrangements of components, and in various steps and arrangements of steps. The drawings are only for purposes of illustrating example embodiments and are not to be construed as limiting the present invention.

FIG. 1 illustrates an infusion reservoir.

FIG. 2 illustrates an alternate infusion reservoir which is implantable within a burr hole and includes an extension for applying medication or allowing passage of an electrode for deep brain stimulation.

FIG. 3 illustrates a catheter tip with capability for perineural insertion.

FIG. 4 is a cross-sectional view of a neurostimulator device in accordance with an aspect of the present invention.

FIG. 5 is a plane view of a neurostimulator device of FIG. 6.

FIG. 6 is a plane view of a burr hole ring with three grooves.

FIG. 7 is a cross-sectional view of a drug delivery device having a receiver configured to wirelessly receive signals and to control dosing of a drug accordingly.

FIG. 8A is a cross-sectional view of an example microinfusion device including a neurostimulator device.

FIG. 8B is a top view of FIG. 8A.

FIG. 9A is a cross-sectional view of an example microinfusion device including a neurostimulator device.

FIG. 9B is a top view of FIG. 9A.

FIG. 10A illustrates an example microinfusion device which is a burr hole ring and includes coils or antennas disposed proximate to the ring-shaped reservoir to provide/receive signals from an external transmitter to the dose controller to control dosing of the drug through the microcatheter.

FIG. 10B depicts a further example of an example device including a neurostimulation electrode having coils/antennas disposed proximate thereto to provide/receive signals from an external transmitter to control neurostimulation, the electrode being inserted via a notch in the reservoir through the burr hole.

FIG. 11 shows a microcatheter device further including a neurostimulator device which is an implantable and includes one or more semiconductor ball implants to provide neurostimulation.

FIG. 12 illustrate a cross-sectional view of a microcatheter device including multiple microcatheters.

FIG. 13 shows a microinfusion device wherein the reservoir has at least one septation to separate different drugs.

### DETAILED DESCRIPTION

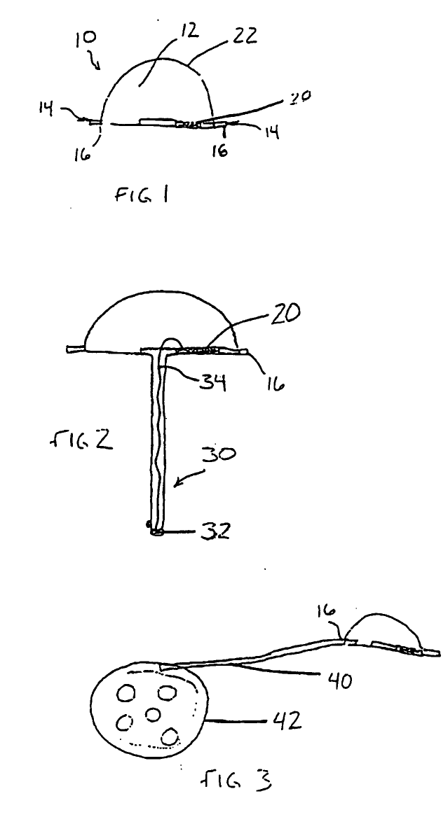

FIG. 1 illustrates a microinfusion device (10) in accordance with an example embodiment of the present invention. In this embodiment, the microinfusion device (10) includes a chamber or reservoir (12) which, in the illustrated embodiment, contains a drug or medication. Here, the reservoir is domed shaped, although other shapes are possible.

The example device further includes a base (14) having a radius larger than the base of the reservoir (12). Incorporated into the base (14) are outlets (16) at opposing sides of the reservoir (12). In alternative embodiments, many outlets may be spaced about the periphery of the base (14). Moreover, in still further embodiments, the base may include only a single outlet (16). The device also includes a dose control (20) which regulates the rate of medication to outlets (16).

In the example embodiment of the present invention illustrated in FIG. 1, an exterior shell (22) of the reservoir (12) includes a penetrable surface such that a hypodermic needle, for example, may be used to deliver or replenish a medication supply without removing the entire device. In an alternate embodiment, the device may be configured as a single use, preloaded system which remains in place until the supply of medication is exhausted.

The device illustrated in FIG. 1 is sized for subcutaneous implantation, e.g., completely under the skin. This example embodiment may also be sized to be mounted within a burr hole of a skull of a subject. Thus, the device may be used for applying a drug to areas within the brain, although other applications are possible.

FIG. 2 depicts another a microinfusion device in accordance with an example embodiment of the present invention. This example embodiment includes a catheter (30), e.g., a microcatheter, coupled to the reservoir. The catheter (30) is configured to deposit or deliver a medication to a target location, such as to a deep or remote location such as in deep brain infusion. A dose control system (20) is also provided which controls the flow of the drug through the catheter (30) and/or outlets (16). The dose control system may include a controller (e.g., an electronic controller) and a valve system (e.g., a micro-valve system). The valve system may be provided between the reservoir and the catheter and/or outlets. In this embodiment, the valve system may be adjustably controlled via an electronic controller; accordingly, the dosing may be adjusted or changed. For example, the electronic controller may wirelessly receive signals (via a receiver) and may control the dose control system in accordance therewith. The signals may be, for example, radio frequency (RF).

The microcatheters coupled to the reservoir will vary in length depending upon the target location to which drugs or other therapeutic substances are to be delivered. For example epidural, subdural, and intraparenchymal/parenchymal microcatheters may be used respectively to deliver medication above the sac, or dura, that covers the brain; below the dura; or into the brain tissue. The microcatheters used for epidural infusion will be the shorter than the other two types of catheters, whereas, intraparenchymal/parenchymal microcatheters will be the longest of the three.

In other embodiments, the valve system may be configured for fixed delivery of the drug for predetermined dosing. Alternatively, the valve system may include a ball bearing system which is magnetically or electronically adjustable for delivery of the drug (e.g., for either fixed or adjustable dosing).

A fiber optic or other sensor (32) may also be included for sensing the medication or other information at the point of interest. Sensed information may be provided to the dose control system (20) via a feedback loop (34). The feedback loop (34) may permit the dose control system (20) to adjust the rate of medication delivery depending on the sensed data. The feedback loop (34) may be a wired or wireless connection.

The sensor (32) may be provided at the distal end of the catheter (30), although other locations are possible and may be desirable. The sensor (32) may detect various physiological parameters, including e.g., intracranial pressure. The device may be configured such that if an intracranial pressure over 15-20 cm water is detected, the dose control system prevents the delivery of the drug for example, by closing valve(s) of the valve system. Another physiological parameter which may be detected by a sensor is intracranial pH. The device may be configured such if an intracranial pH of between 7.3 and 7.5 is detected, the dose control system prevents delivery of the drug. The sensor (32) may also detect physiological parameters selected from the group consisting of pO2, pCO2, glucose concentration, lactic acid concentration, or concentration of the delivered drug. In an example configuration, detection by the sensor (32) of excessive or insufficient partial pressure of oxygen or carbon dioxide or both, excessive or insufficient concentration of glucose, lactic acid, or the delivered drug, or any combination thereof, provides a signal to the dose control system to prevent delivery of the drug.

The catheter (30) of FIG. 2 may have multiple ports for drug delivery. It may also include a recording/multi-contact stimulating electrode, as described by in U.S. Pat. No. 5,676,655, issued on Oct. 14, 1997 to Howard, III et al., which is hereby incorporated by reference herein in its entirety.

In another embodiment, the microinfusion device shown in FIG. 2 may include more than one catheter. This embodiment may be advantageously in situations where it is desirable (or necessary) to deliver drugs to two different target locations. In such an embodiment, each catheter may have a sensor associated therewith, such as the sensor (30) described above. Drugs flowing through these catheters may be controlled by one dose control system, or individual control systems.

Referring now to FIG. 3, the device is shown with a catheter (40) connected to an outlet (16). The catheter (40) here may be inserted into an area of interest, such as a peripheral nerve (42) as illustrated.

FIG. 13 illustrates another example embodiment in accordance with the present invention. In this device, the reservoir includes one or more septations (35) to separate different drugs within the reservoir. This provides a means to introduce multiple different drugs into the central or peripheral nervous system via one or more catheters and/or one or more outlets. An adjustable valve may be provided in each compartment separated by the septation(s) 35, and the valve(s) may be configured to deliver the drugs to the microcatheter and may be controlled by the dose controller (20), and a sensor (32) configured to the dose controller which may provide a feedback loop to control drug dosing.

The microinfusion devices described herein may, in some embodiments, be attached to a burr hole device, such as a burr hole ring. Reference is made herein to U.S. Pat. No. 6,044,304 and U.S. Patent Publication No. 2002/0052610 published on May 2, 2002, each of which is expressly incorporated herein by reference in its entirety.

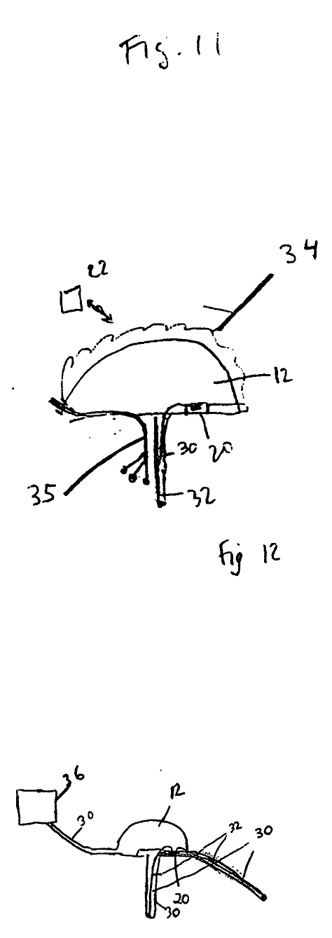

In an alternate embodiment, the microinfusion device comprises the actual burr hole ring, thereby obviating the need to use burr hole devices, as shown in FIGS. 10A and 10B. For example, the circle of the ring will include a chamber or reservoir with a port for injection and a tab or notch for insertion of a cannula and may also include a notch for insertion of an electrode, as described above. Burr hole ring devices which have previously been described may be modified for use with the devices provided herein. For example, U.S. Pat. No. 5,954,687, issued on Sep. 21, 1999 to Baudino, which is hereby incorporated herein by reference in its entirety, describes a burr hole ring with a catheter for use as an injection port, whose burr hole ring interior serves as a reservoir and is in fluid communication with a catheter. Likewise, a surgically implantable burr-hole flow control device positionable upon a burr hole for controlling proximal-to-distal flow of cerebrospinal fluid from brain ventricles to another portion of the body has been described in U.S. Pat. No. 5,800,376, issued on Sep. 1, 1998 to Watson et al., which is hereby incorporated by reference in its entirety herein.

The microinfusion device provided herein in its simplest form may be a disposable system for fixed dosing a single medication or drug which could be implanted in a patient as a tool for trial chemical modulation. Prior to the present invention, this was accomplished through the implantation of a large and bulky drug delivery system having a diameter of about 7.5 cm.

In other forms, the device is semi-permanent and reusable but still more compact than present drug delivery systems. For example, if a trial of a drug delivered with the disposable device achieves the desired effect(s) on the target location(s), a semi-permanent and reusable device may be implanted in the skull of the subject for an extended time period, e.g., to continue delivery of the drug in doses that achieve the successful effect on the target location.

In example embodiments, the compact size of either the disposable or semi-permanent devices may be advantageous when delivering a drug or chemical within the substrate of the central or peripheral nervous system. Such systems permit dosing concentrations for direct nervous system injection that are an order of magnitude smaller than either oral, intravenous, or intrathecal dosages.

In example embodiments of the above-described devices, the target location is in the nervous system of the subject, for example the central nervous system (e.g., the brain), the peripheral nervous system, systemic nervous tissue or the spinal cord. In other example embodiments, the device allows the delivery of multiple types of drugs, chemicals, medication and the like, at a controlled rate of delivery. As described above, the device may be subcutaneously implantable. The device can be utilized for delivery of drugs, chemicals, gene therapy vectors, viral vectors into the central or peripheral nervous system. Additionally, the device may be used for dosed delivery of chemotherapy or antibiotic(s) over the course of many days to weeks.

The applications of the example microinfusion devices of the present invention include (but are not limited to) drug delivery for Parkinson's Disease Essential tremor, MS, Dystonia, cerebral palsy, psychiatric disorders, obsessive compulsive disorder, depression, ALS and gene therapy vectors to allow delivery of a substance retrograde through the peripheral nerves. The example devices may also be used for controlled antibiotic therapy for meningitis, bacterial or chemical. A further use of the microinfusion device includes delivery of chemotherapy for carcinomatous meningitis, central nervous system lymphoma or other metastatic disease. The microinfusion devices may also be used to deliver other agents or therapeutic substances, for example hot or cold saline may be infused for the treatment of epilepsy.

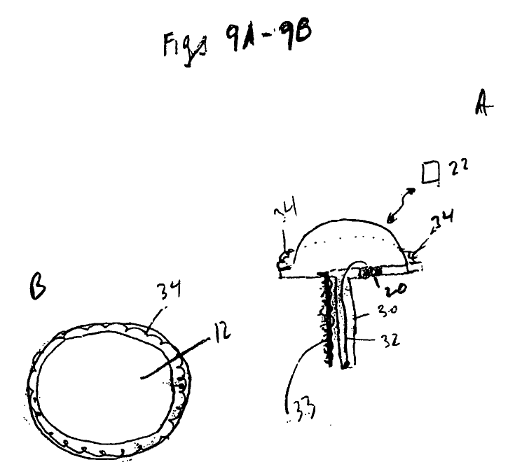

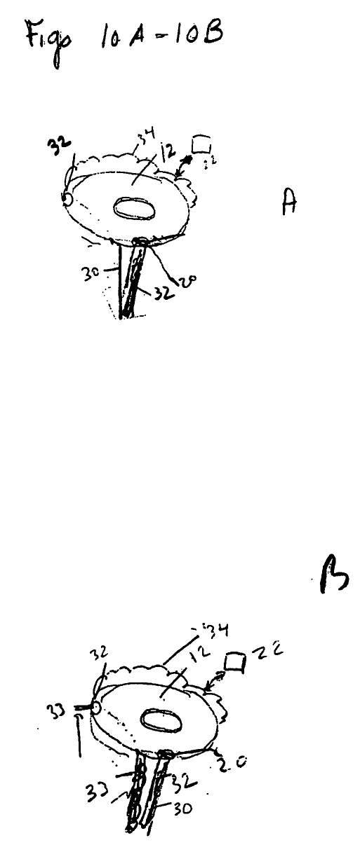

FIG. 7 illustrates a drug delivery device (e.g., microinfusion device) having a dose control system (20), a sensor (32) and a receiver and/or transmitter (34) including, for example, coils and/or antennas which receive signals from an external transceiver (22). In this embodiment, the receiver (34) is subcutaneously implanted, along with the drug delivery device (such as the devices described above). In one embodiment, the transceiver (22) transmits signals wirelessly (e.g., RF signals) to the receiver (34). Received signals are used by the dose control system (20) to control the dosing of the drugs in the reservoir. For example, the received signals may provide pulses for controlling the dose control system. In other embodiments, the received signals may include parameter information with which modifications to a controller of the dose control system may be made. The receiver/transmitter (34) may also be configured to transmit information sensed by the sensor 32 to the external transceiver (22). U.S. Published Patent Application No. 2002/0091419, published on Jul. 11, 2002 (which is expressly incorporate herein by reference, in its entirety, and particularly FIG. 9 thereof) shows one receiver/transmitter arrangement that may be modified for use in connection with the drug delivery system described herein.

In FIG. 7, the receiver (34) is implanted proximate to the drug delivery device. In other embodiments, the receiver may be integral with the device, or may be implanted at a distance from the drug delivery device.

FIGS. 8A and 8B illustrate another example of a microinfusion device which may further include a neurostimulator device. The neurostimulator device (33) (including, for example, a deep brain (DBS) electrode) may receive signals from and/or transmit signals to an external transmitter (22) via radio frequency coils/antennas (34), which are disposed proximate to the neurostimulation electrode, and/or also via radio frequency coils (34) disposed proximate the reservoir (12), to control dosing of a drug from the reservoir. A lead (26) may connect the external RF coils to the electrode. Accordingly, in this embodiment, both the neurostimulator device (33) and the dosing of drugs may be controlled wirelessly, via RF signals, for example. FIGS. 9A and 9B illustrate yet another example microinfusion/neurostimulator device. In this embodiment, the coils/antennas (34) are disposed proximate to the base of the device.

FIGS. 10A and 10B illustrate another example embodiment of a microinfusion device which is a burr hole ring. This example device may include coils or antennas (34) disposed proximate to the ring-shaped reservoir (12) to transmit and/or receive signals from an external transmitter (22) to the dose controller (20) to control dosing of the drug through the microcatheter (30). A sensor (32) configured to the dose controller (20) provides a feedback loop. In another example embodiment, the microinfusion device may be configured with a neurostimulation electrode (33), as shown in FIG. 10B, which may also include coils and/or antennas disposed proximate thereto to transmit and/or receive signals from an external transmitter (22) to control neurostimulation, wherein the electrode may be inserted via a notch (32) in the reservoir (12) through the burr hole. The receiver (34) may be an active or a passive device.

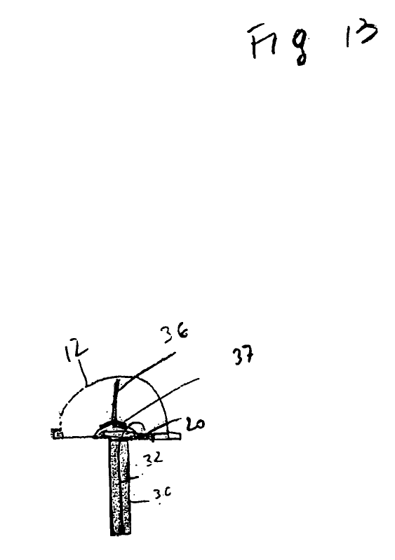

FIG. 11 shows a microinfusion/neurostimulation device according to an example embodiment of the present invention, wherein the device may include a sensor (32), a dose controller (20), a neurostimulator device which is implantable and includes one or more semiconductor ball implants (35) to provide neurostimulation. Signals are received from and/or transmitted to an external transmitter (22) via radio frequency coils (34) disposed proximate to the neurostimulator device and via radio frequency coils (34) disposed proximate to the base of the device, respectively, to control dosing of a drug from the reservoir (12).

FIG. 12 depicts a microinfusion device in another example embodiment of the present invention, wherein the example device includes multiple microcatheters (30), wherein one microcatheter (30) connects the reservoir (12) of the infusion device to an second larger reservoir (36) implanted within a body of a subject, which second reservoir (36) is a source of the drug to be delivered. One or more additional microcatheters (30) connected to the microcatheter reservoir (12) may be configured to deliver drugs to target locations. The device may also include a receiver (and/or transmitter), e.g., RF coils or antennas (34) disposed proximate to the reservoir (12) which may receive and/or transmit signals to the dose controller to control drug flow through the microcatheter.

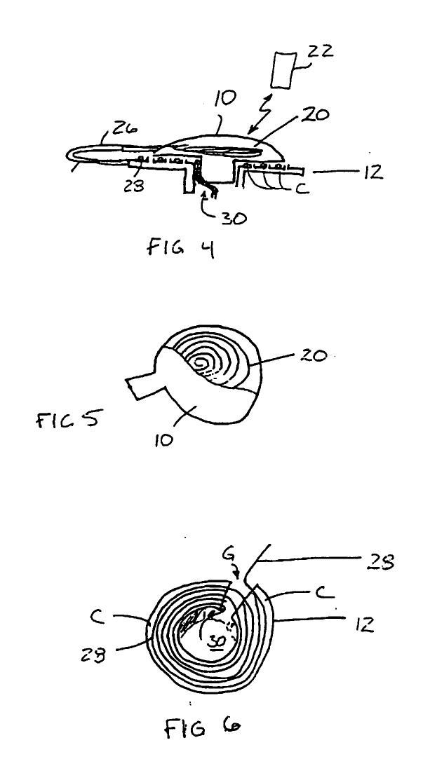

FIG. 4 illustrates a neurostimulation device according to one embodiment of the present invention. In this embodiment, the neurostimulation device may include a cap (10) which rests snugly in a burr hole ring (12) overlaying a region of interest. Disposed within cap (10) are antenna windings (20) which are suitable for receiving RF radiation from a transmitter (22). A lead (26) from winding (20) connects with windings (28) associated with burr ring (12). In the illustrated embodiment, burr ring (12) includes a plurality of channels (C) in which the windings reside. At an end of channel (C) towards central opening (30) the implanted electrode is electrically connected to the winding (28). This completes the electrical connection between the coil or winding (20) and the electrode tip disposed in the treatment site (not shown).

In one example embodiment of the neurostimulation device, the RF coil(s) is sized so as to be implanted in the subgaleal space or other locations susceptible to external stimulation. In another aspect, the device may include a small temporary impulse generator coupled to a previously implanted neurostimulation electrode. The small temporary impulse generator may be implanted in a location conducive to external, that is outside the skin, stimulation. In accordance with another aspect of the present invention, the neurostimulator device may include a burr hole with a number of grooves. In another embodiment of the neurostimulation device, the burr hole ring cap includes RF coils. In a further embodiment, the RF coils are sealed in a discrete compartment which can be tunneled at a site distance to the burr hole. In accordance with another aspect of the present invention, the device includes an external transmitting assembly. The device may further include an extension system to connect to a single or, alternately, a plurality of electrodes.

Another example embodiment of the present neurostimulation device includes a set of compact RF compatible receiving coils or a smaller temporary impulse generator which is coupled to an already implanted deep brain stimulator electrodes or any other neurostimulation electrode such as that which is used for motor cortex or spinal cord stimulation. This compact receiving RF coil or smaller temporary impulse generator may be implanted in the subgaleal space and may be externally stimulated with an accessory external antenna (outside of the skin) connected to a battery powered transmitter (in the case of an RF system).

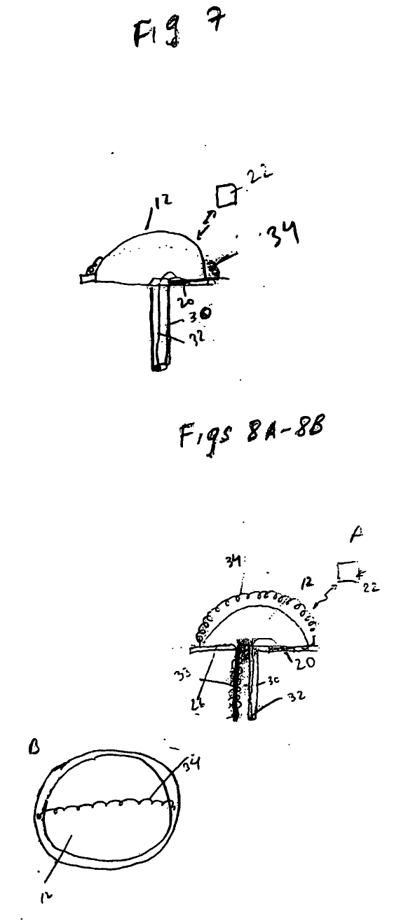

In FIG. 5, a plan view of the cap (10) is illustrated partially cut away to reveal windings (20) disposed therein. The cap is may be made from a material which will readily pass the received RF energy to the coil or windings (20) disposed within and may be implanted subcutaneously.

In FIG. 6, a plan view of the burr ring (12) reveals a channel (C) through which the winding (28) is wrapped. The ring has a gap (G) permitting, among other things, custom and secure fit within the burr hole.

In an alternative embodiment of the devices described above, the neurostimulator device (33) may include one or more semiconductor ball implants, rather than, for example, a DBS electrode. An implantable neurostimulator with one or more semiconductor ball implants is described in U.S. Pat. No. 6,415,184, issued on Jul. 2, 2002 to Ishikawa et al., which is expressly incorporated herein by reference in its entirety.

In this example embodiments described above, its smaller size may allow for its implantation into the head or via a small incision after the insertion of a percutaneous spinal cord stimulator system. Additionally, it may be cheaper and smaller than the currently available totally implantable pulse generators (IPGs). The neurostimulation devices provided herein may be implanted to allow a patient with a deep brain stimulation electrode to trial the effects of stimulation especially in the case of pain, psychiatric disorders, dystonia, addictions, brain injury, or epilepsy, where the benefits of stimulation are not anticipated to occur for days to few months. The costliest part of a current deep brain stimulation procedure may be the cost of the IPGs. One RF device may run both coils and electrodes. This arrangement may obviate the need for externalized wires for trial testing.

This neurostimulation devices described herein may have wide application including the entire neurostimulation spectrum for pain, as well as all of the emerging applications of brain stimulation for which the efficacy has yet to be been fully determined. The devices may be used for neurostimulation in dystonia, psychiatric disorders such as OCD, depression, schizophrenia, epilepsy, traumatic brain injury, morbid obesity, etc. In these common scenarios, there could be a substantial cost incurred to the medical industry and society if each of these patients were to receive the currently used totally implantable pulse generator (IPG) costing $10,000 each. The current system provided by the present invention may obviate another surgical procedure and such an up-front cost, which in most circumstances would include two impulse generators for bilateral or system type disease.

The present invention has been described with reference to example embodiments. However, mdifications and alterations will occur to others upon reading and understanding the preceding Detailed Description. For example, various portions of the example embodiments may be combined in ways other than expressly described herein above.

1. A microinfusion device, comprising:
a) a subcutaneously implantable reservoir configured to contain a drug, the reservoir being mountable within a burr hole of a skull of a subject;
b) a dose control system configured to control flow of the drug; and
c) a microcatheter configured to deliver the drug from the reservoir to a target location.

2. The device of claim 1, wherein the reservoir is mounted within a burr hole ring.

3. The device of claim 1, wherein the dose control system includes a valve system between the reservoir and the microcatheter for flowing of the drug.

4. The device of claim 3, wherein the valve system is fixed for delivery of the drug.

5. The device of claim 3, wherein the valve system is electronically adjustable to control delivery of the drug.

6. The device of claim 1, wherein the dose control system includes a ball-bearing system, the ball-bearing system being magnetically adjustable for delivery of the drug.

7. The device of claim 6, wherein the ball-bearing system is electronically adjustable to control delivery of the drug.

8. The device of claim 1, further comprising:
a sensor at the distal end of the microcatheter to provide feedback to the dose control system.

9. The device of claim 1, further comprising:
radio frequency coils or antennas configured to receive signals from an external controller, the dose control system controlling the flow of the drug in accordance with the received signals.

10. The device of claim 1, wherein the reservoir includes a penetrable surface for delivery of the drug to the reservoir with a hypodermic needle.

11. The device of claim 1 which is a disposable device for fixed dosing of one drug.

12. The device of claim 1, wherein the reservoir includes at least one septation to separate different drugs within the reservoir.

13. The device of claim 1, wherein the reservoir includes multiple septations to separate multiple drugs within the reservoir.

14. The device of claim 1 which is a semi-permanent and reusable device.

15. The device of claim 1, wherein the target location is in the nervous system of the subject.

16. The device of claim 15, wherein the nervous system is the central nervous system, the peripheral nervous system, systemic nervous tissue or the spinal cord.

17. A microinfusion device, comprising:
a subcutaneously implantable reservoir configured to contain a drug, the implantable reservoir having at least two outlets.

18. The device of claim 17, wherein the reservoir is configured to engage a burr hole in a skull of a subject.

19. The device of claim 17, wherein the reservoir is mountable within a burr hole ring in a skull of a subject.

20. The device of claim 17, further comprising:
a dose control system configured to control flow of the drug.

21. The device of claim 20, wherein the dose control system includes a valve system between the reservoir and the microcatheter for flowing of the drug.

22. The device of claim 21, wherein the valve system is fixed for delivery of the drug.

23. The device of claim 21, wherein the valve system is electronically adjustable to control delivery of the drug.

24. The device of claim 20, wherein the dose control system includes a ball-bearing system, the ball-bearing system being magnetically adjustable for delivery of the drug.

25. The device of claim 24, wherein the ball-bearing system is electronically adjustable to control delivery of the drug.

26. The device of claim 17, further comprising:
respective microcatheters connected to each of the at least two outlets, wherein at least one of the respective microcatheters is connected to a reservoir infusion system, said reservoir infusion system being implantable within the body of a subject to provide a source of the drug, and the at least another of the respective microcatheters is configured to deliver the drug to a target location.

27. The device of claim 26, further comprising:
a sensor at the distal end of the at least second microcatheter to provide feedback to the dose control system.

28. The device of claim 27, wherein the sensor detects intracranial pressure.

29. The device of claim 27, further comprising:
radio frequency coils or antennas configured to receive signals from an external controller, the dose control system controlling the flow of the drug in accordance with the received signals.

30. The device of claim 17, wherein the reservoir includes a penetrable surface which permits delivery of the drug to the reservoir with a hypodermic needle.

31. The device of claim 17 which is a semi-permanent and reusable device.

32. The device of claim 17, wherein the reservoir includes at least one septation to separate different drugs within the reservoir.

33. The device of claim 17, wherein the reservoir includes multiple septations to separate multiple drugs within the reservoir.

34. The device of claim 17, wherein the target location is in the nervous system of the subject.

35. The device of claim 34, wherein the nervous system is the central nervous system, the peripheral nervous system, systemic nervous tissue or the spinal cord.

36. A microinfusion device, comprising:
a) a subcutaneously implantable reservoir configured to contain a drug, the reservoir being mountable within a burr hole in a skull of a patient, the reservoir being ring-shaped; and
b) a microcatheter configured to deliver the drug from the reservoir to a target location.

37. The device of claim 36, wherein the reservoir is mountable within a burr hole ring.

38. The device of claim 36, further comprising:
a dose control system configured to control flow of the drug.

39. The device of claim 38, wherein the dose control system includes a valve system between the reservoir and the microcatheter for flowing of the drug.

40. The device of claim 39, wherein the valve system is fixed for delivery of the drug.

41. The device of claim 39, wherein the valve system is electronically adjustable to control delivery of the drug.

42. The device of claim 38, wherein the dose control system includes a ball-bearing system, the ball-bearing system being magnetically adjustable for delivery of the drug.

43. The device of claim 42, wherein the ball-bearing system is electronically adjustable to control delivery of the drug.

44. The device of claim 36, further comprising:
a sensor at the distal end of the microcatheter to provide feedback to the dose control system.

45. The device of claim 57, wherein the sensor detects intracranial pressure.

46. The device of claim 36, further comprising:
radio frequency coils or antennas configured to receive signals from an external controller, the dose control system controlling the flow of the drug in accordance with the received signals.

47. The device of claim 36, wherein the ring-shaped reservoir includes a penetrable surface to deliver the drug to the reservoir with a hypodermic needle.

48. The device of claim 36 which is a disposable device for fixed dosing of one drug.

49. The device of claim 36, wherein the ring-shaped reservoir includes at least one septation to separate different drugs within the reservoir.

50. The device of claim 36, wherein the ring-shaped reservoir includes multiple septations to separate multiple drugs within the reservoir.

51. The device of claim 36, further comprising:
the reservoir having at least one notch for insertion of a deep brain stimulation electrode through the at least one microcatheter.

52. The device of claim 36 which is a semi-permanent and reusable device.

53. The device of claim 36, wherein the target location is in the nervous system of the subject.

54. The device of claim 53, wherein the nervous system is the central nervous system, the peripheral nervous system, systemic nervous tissue or the spinal cord.

55. A microinfusion device, comprising:
a) a subcutaneously implantable reservoir having septations configured to separate different drugs within the reservoir; and
b) at least one microcatheter configured to deliver the different drugs from the reservoir to at least one target location.

56. The microinfusion device of claim 55, wherein the reservoir is mountable within a burr hole of a skull of a subject.

57. The microinfusion device of claim 55, wherein the reservoir is mounted within a burr hole ring.

58. The microinfusion device of claim 55, further comprising:
a dose control system configured to control flow of the drug.

59. The device of claim 58, wherein the dose control system includes a valve system between the reservoir and the microcatheter for flowing of the drug.

60. The device of claim 59, wherein the valve system is fixed for delivery of the drug.

61. The device of claim 59, wherein the valve system is electronically adjustable to control delivery of the drug.

62. The device of claim 58, wherein the dose control system includes a ball-bearing system, the ball-bearing system being magnetically adjustable for delivery of the drug.

63. The device of claim 62, wherein the ball-bearing system is electronically adjustable to control delivery of the drug.

64. The device of claim 58, further comprising:
a sensor at the distal end of the at least one microcatheter to provide feedback to the dose control system.

65. The device of claim 64, wherein the sensor detects intracranial pressure.

66. The device of claim 55, further comprising:
radio frequency coils or antennas configured to receive signals from an external controller, the dose control system controlling the flow of the drug in accordance with the received signals.

67. The device of claim 55, wherein the reservoir includes a penetrable surface for delivery of the drug to the reservoir with a hypodermic needle.

68. The device of claim 55 which is a disposable device for fixed dosing of one drug.

69. The device of claim 55, wherein the reservoir includes at least one septation to separate different drugs within the reservoir.

70. The device of claim 55, wherein the reservoir includes multiple septations to separate multiple drugs within the reservoir.

71. The device of claim 55 which is a semi-permanent and reusable device.

72. The device of claim 55, further comprising:
the reservoir having at least one notch for insertion of a deep brain stimulation electrode through the at least one microcatheter.

73. The device of claim 55, wherein the target location is in the nervous system of the subject.

74. The device of claim 73, wherein the nervous system is the central nervous system, the peripheral nervous system, systemic nervous tissue or the spinal cord.

75. A drug delivery device, comprising:
a) a reservoir configured to contain a drug; and
b) a receiver configured to wirelessly receive signals and to control a dosing of the drug in accordance therewith.

76. The drug delivery device according to claim 75, wherein the signals are radio frequency signals.

77. The drug delivery device according to claim 75, wherein the reservoir is subcutaneously implantable.

78. The drug delivery device according to claim 77, further comprising:
dose control system configured to control the dosing of the drug as a function of the signals.

79. The drug delivery device according to claim 78, wherein the dose control system includes a valve system.

80. The drug delivery device according to claim 77, further comprising:
a microcatheter coupled to the reservoir and configured to deliver the drug to the target location.
